(19) United States
(12) Patent Application Publication
(10) Pub. No.: US 2009/0131493 A1
(43) Pub. Date: May 21, 2009

(54) SUBSTITUTED BENZIMIDAZOLES FOR TREATMENT OF HISTOMONIASIS

(75) Inventors: Gisela Greif, Remagen (DE); Robrecht Froyman, Monheim (DE)

Correspondence Address: BAYER HEALTHCARE LLC, P.O.BOX 390, SHAWNEE MISSION, KS, 66201, US

(73) Assignee: BAYER HEALTHCARE AG, D-51368 LEVERKUSEN (DE)

(21) Appl. No.: 11/813,354

(22) PCT Filed: Dec. 29, 2005
(86) PCT No.: PCT/EP2005/014119

§ 371 Date: Dec. 23, 2008

(30) Foreign Application Priority Data
Jan. 5, 2005 (DE) 10 2005 000 746.5

(51) Int. Cl. A61K 31/4184

(52) U.S. Cl. 514/394

(57) ABSTRACT

The present invention relates to the use of substituted benzimidazoles for controlling histomoniasis, especially in turkeys.

The present invention relates to the use of substituted benzimidazoles for treating histomoniasis in poultry, especially in turkeys.

Substituted benzimidazoles and their use as insecticides, fungicides and herbicides have previously been disclosed (EP-A 87 375, 152 360, 181 826, 239 508, 260 744, 266 984, U.S. Pat. Nos. 3,418,318, 3,472,865, 3,576,818, 3,728,994). Halogenated benzimidazoles and their effect as anthelmintics, coccidiostats and pesticides have been disclosed (DE-A 2 047 369, DE-A 4 237 617). The substituted benzimidazoles which are preferably employed according to this invention are described in WO 00/04022 and WO 00/68225.

Mixtures of nitro-substituted benzimidazoles and polyether antibiotics have been disclosed as remedies for coccidiosis (U.S. Pat. No. 5,331,003). Mixtures of substituted benzimidazoles with polyether antibiotics or synthetic remedies for coccidiosis as compositions for controlling parasitic protozoa are disclosed in WO 96/38140.

Histomoniasis (“blackhead disease”) is an infectious disease. It is caused by Histomonas spp., especially Histomonas meleagridis, which is one of the intestinal parasites. The infection leads to a severe inflammation of the caecum and liver because the pathogen damages intestinal tissue and reaches the liver via the blood and causes necroses to form there. A frequent concomitant effect of the disease is circulatory failure, evident from the blackish-blue heads of diseased birds, from which the name of the disease derives.

In infected farms, the disease very rapidly spreads to the entire stock and leads, owing to the very high mortality rates (up to 100% in some farms), to great economic losses.

Histomonas meleagridis belongs, because of its structural flagella, to the subphylum of flagellates (Mastigophora). The flagellate stages multiply in the caecum by dividing into two. Amoeboid-like stages originating in the infected caecum invade the liver via the bloodstream and destroy it by extensive necroses (BonDurant, R. H., Wakenell, P, S. (1994): Histomonas meleagridis and Relatives. In: Parasitic Protozoa, Volume 9, Chapter 3, pp 189-206. Academic Press)

Direct transmission of histomonads, e.g. oral intake of histomonad-containing fresh faeces, always fails because the pathogens are able to live for only a short time outside a host and are killed on passing through the digestive tract. Tests by American researchers (Hu and McDougald, 2003) revealed experimentally that cloacal infection is possible in turkeys. Since the cloaca generates a slight suction after deposition of faeces, infection of a flock by this route is probable under practical conditions, e.g. through soiled bedding. It has also been scientifically demonstrated that the pathogen is transmitted by intermediate hosts. The caecal worm Heterakis gallinarum is known to be a carrier (transport vector of Histomonas meleagridis), especially the heterakis eggs or larval stages. This is why chickens and turkeys may already be actively infected by histomonads and become ill even before adult heterakis worms appear in the caecum contents. Histomonads may stay infectious for up to 4 years in embryonate heterakis eggs. Further intermediate hosts may be earthworms and arthropods contaminated with heterakis eggs. Chickens and other types of poultry also represent a potential risk. They are less sensitive than turkeys and are often carriers of the pathogen without clinical manifestations, so that they contribute to spreading the pathogen.

Turkeys may be infected at any age, but the disease occurs most often between week 3 and 12 of life. The period between infection and appearance of the disease is usually 7-12 days. Mortality may be up to 100% and reaches its highest level on day 17 after infection. Inflammations in the caecum are to be found from day 8, and in the liver from day 10 onwards.

Infected birds are listless and exhausted, show drooping wings and head and refuse food. Sulphur-yellow droppings, diarrhoea and later also the presence of blood are typical. The circulatory impairments associated with the disease cause a pronounced blackish-blue colour of the head, whence the name of this disease.

The progress of the disease is determined primarily by age and intestinal flora of the turkeys. Additional bacterial infections with E. coli, Clostridium perfringens or coccidia make the course more severe (McDougald, L. R., Hu, J. (2001): Blackhead Disease (Histomonas meleagridis) aggravated in broiler chickens by concurrent infection with cecal coccidiosis (Eimeria tenella). Avian Diseases 45:307-312)

The disease can be diagnosed from specimens taken from the caecum and liver with the aid of a saline solution. Stages showing amoeboid movement are visible under a phase-contrast microscope. The PAS stain is used for histology.

Until 1950, arsenic compounds (e.g. nitarsone, carbarsone, roxarsone) were the only compounds effective for histomoniasis. However, it is known that arsenic compounds are generally not strong enough for treating infections once established. A further disadvantage is their extremely low safety margin; just doubling the dosage of roxarsone leads to impaired motor functions in the turkey cock.

Since 1960, nitroimidazoles and nitrofurans have been employed intensively in the feed and/or water, but use in productive livestock and as feed additive was banned increasingly by the EU and the USA from the mid-1990s: dimetridazole was removed from the market in the USA in 1997 and was banned for use as feed additive in the EU in 2001. Since 31 Mar. 2003 it has no longer been possible to employ nifursol, the only product still authorized in the EU, either. Thus, neither medicaments for therapy nor products for the prophylactic control of blackhead disease are available now and in the future.

Helminth-active substances (albendazole and fenbendazole) have inadequate activity on histomonas in vitro, but have prophylactic activity in vivo if treatment takes place for 14 days after the time of infection. The activity in this case is directed not against H. meleagridis but against Heterakis gallinarum, the transport vector of Histomonas meleagridis (Hu, J., McDougald, L. R., (2003): Direct lateral transmission of Histomonas meleagridis in turkeys. Avian Diseases 47:489-492).

The only strategies available at present for preventing disease consist of hygiene measures, optimization of the stocking density and nutrient supply, and prevention of pathogen transmission. These measures are inadequate and cannot prevent infection and disease.

Vaccination against Histomonas meleagridis is not biologically possible because natural immunity cannot be acquired after infection either. Birds infected once can become ill again. Trials with immunization using attenuated live vaccines had no success.

There is hence a need for agents for treating histomoniasis.

The invention relates to the use of benzimidazoles of the formula (I)

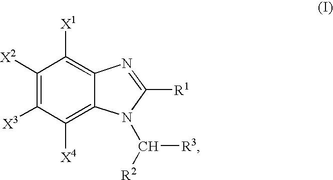

in which
R¹ is fluoroalkyl,
R² is hydrogen or alkyl,
R³ is a radical of the formula

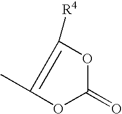

or is a radical of the formula

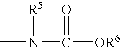

R⁴ is alkyl,
R⁵ is alkyl or substituted phenyl,
R⁶ is alkyl,
X¹, X², X³ and X⁴ are independently of one another hydrogen, halogen, haloalkyl, haloalkoxy, haloalkylthio or haloalkylsulphonyl,
   or else
X² and X³ or X³ and X⁴ together are a dioxyhaloalkylene radical,
   for manufacturing medicaments for controlling histomoniasis in poultry.

The substituted benzimidazoles of the invention are defined generally by the formula (I).
R¹ is preferably C₁-C₄-fluoroalkyl,
R² is preferably hydrogen or C₁-C₄-alkyl,
R⁴ is preferably C₁-C₄-alkyl,
R⁵ is preferably C₁₋₆-alkyl or phenyl which is optionally substituted one or more times by C₁₋₄-alkyl, C₁₋₄-haloalkyl, halogen, nitro, C₁₋₄-alkoxy, C₁₋₄-haloalkoxy or optionally mono- or poly-halogen-substituted methylene- or ethylenedioxy.
R⁶ is preferably C₁₋₄-alkyl
X¹, X², X³ and X⁴ are preferably independently of one another hydrogen, F, Cl, Br, C₁-C₄-haloalkyl, C₁-C₄-haloalkoxy, C₁-C₄-haloalkylthio, C₁-C₄-haloalkylsulphonyl, or
X² and X³ or X³ and X⁴ are in a further preferred embodiment together a dioxyhalo-C₁-C₄-alkylene radical.
R¹ is particularly preferably CF₃, CHF₂ or CHF.
R² is particularly preferably hydrogen, methyl, ethyl, n-propyl or isopropyl.
R⁴ is particularly preferably methyl, ethyl, n-propyl or isopropyl.
R⁵ is particularly preferably C₁₋₆-alkyl.
R⁶ is particularly preferably methyl or ethyl.
X¹, X², X³ and X⁴ are particularly preferably independently of one another hydrogen, F, Cl, Br, CF₃, CHF₂, CH₂F, OCF₃, OCH₂F, OCHF₂, SCF₃, SCHF₂, SCH₂F, SO₂CF₃, SO₂CHF₂, SO₂CH₂F.
X² and X³ or X³ and X⁴ are in a further embodiment particularly preferably also together a radical —O—CF₂—O—, —O—CF₂—CF₂—O—, —O—CF₂—CF₂—CF₂—O—, —O—CF₂—CHF—O—, —O—CClF-CClF-O—, —O—CHF—O—, —O—CHF—CHF—O— or —O—CClF-O—.

In a very particularly preferred embodiment, R³ is a radical of the formula

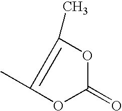

In a further very particularly preferred embodiment, R³ is a radical of the formula

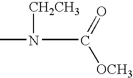

R¹ is very particularly preferably —CF₃.
R² is very particularly preferably hydrogen.
R⁴ is very particularly preferably methyl.
X¹ is very particularly preferably Cl or Br.
X² is very particularly preferably hydrogen.
   and
X³ and X⁴ are very particularly preferably together —OCF₂—CF₂—O—.

Alkyl is a straight-chain or branched hydrocarbon radical having 1 to 8, preferably 1 to 6, particularly preferably 1 to 4, carbon atoms, such as, for example, methyl, ethyl, propyl, isopropyl, n-butyl, sec-butyl, tert-butyl.

Alkylene is a straight-chain or branched hydrocarbon radical having 1 to 4, preferably 1 to 3, particularly preferably 1 to 2, carbon atoms, which is linked by two different positions.

Haloalkyl is an alkyl radical as defined above in which one or more, in particular 1 to 3, hydrogen atoms have been replaced by a halogen atom, in particular fluorine, chlorine or bromine.

Fluoroalkyl radical is correspondingly an alkyl radical in which 1 to all hydrogen atoms have been replaced by fluorine atoms; perfluoroalkyl radicals, e.g. trifluoromethyl or pentafluoroethyl, are preferred.

Haloalkoxy is a straight-chain or branched alkoxy radical having 1 to 8, preferably 1 to 6, particularly preferably 1 to 4, carbon atoms, in which one or more, in particular 1 to 3, hydrogen atoms have been replaced by a halogen atom, in particular fluorine, chlorine or bromine; e.g. —OCF₃.

Haloalkylthio is a straight-chain or branched alkylthio radical having 1 to 8, preferably 1 to 6, particularly preferably 1 to 4, carbon atoms, in which one or more, in particular 1 to 3, hydrogen atoms have been replaced by a halogen atom, in particular fluorine, chlorine or bromine; e.g. CF₃S—.

Haloalkylsulphonyl is a straight-chain or branched alkylsulphonyl radical having 1 to 8, preferably 1 to 6, particularly preferably 1 to 4, carbon atoms, in whose alkyl moiety one or more, in particular 1 to 3, hydrogen atoms have been replaced by a halogen atom, in particular fluorine, chlorine or bromine.

Examples of substituted benzimidazoles which are particularly preferred according to the invention are the compound of the formula (I-1) (see WO 00/04022) and in particular the compound of the formula (I-2) (see WO 00/68225):

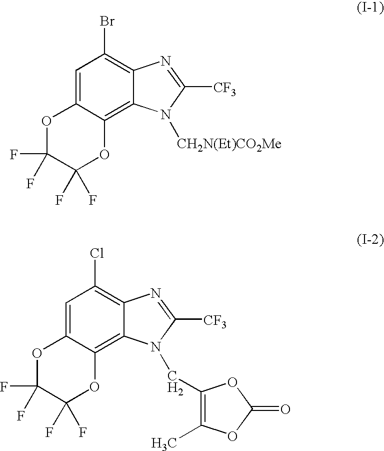

The aforementioned active ingredients may where appropriate, depending on the nature and number of the substituents, be in the form of geometric and/or optical isomers or regioisomers or their isomer mixtures in varying composition. Both the pure isomers and the isomer mixtures can be employed according to the invention.

Where the active ingredients are able to form salts, use in the form of pharmaceutically acceptable salts is also suitable.

The use of hydrates or other solvates of the active ingredients or their salts is also suitable where appropriate.

Both prophylactic and therapeutic use is possible.

Histomoniasis is caused by Histomonas spp. The histomoniasis preferably controlled according to the invention is that caused by Histomonas meleagridis.

The treatment according to the invention is normally applied to poultry such as, for example, chickens, quail, ducks, geese, pheasants and in particular turkeys (here synonymous with turkey cocks).

The active ingredients are used enterally, parenterally, dermally, directly or in the form of suitable preparations.

Enteral use of the active ingredients takes place for example orally in the form of powders, suppositories, tablets, capsules, pastes, drinks, granules, drenches, boluses, medicated feed or drinking water. Cutaneous use takes place for example in the form of dipping, spraying, bathing, washing, pouring on and spotting on, and dusting. Parenteral use takes place for example in the form of injection (intramuscular, subcutaneous, intravenous, intraperitoneal) or by implants.

Suitable preparations are:

solutions such as solutions for injection, oral solutions, concentrates for oral administration after dilution, solutions for use on the skin or in body cavities, pour-on formulations, gels;
emulsions and suspensions for oral or cutaneous use and for injection; semisolid preparations.

Formulations in which the active ingredient is processed in an ointment base or in an oil-in-water or water-in-oil emulsion base;

solid preparations such as powders, premixes or concentrates, granules, pellets, tablets, boluses, capsules; aerosols and inhalations, active-ingredient-containing mouldings.

Solutions for injection are administered intravenously, intramuscularly and subcutaneously.

Solutions for injection are prepared by dissolving the active ingredient in a suitable solvent and possibly adding additions such as solubilizers, acids, bases, buffer salts, antioxidants, preservatives. The solutions are sterilized by filtration and bottled.

Solvents which may be mentioned are: physiologically tolerated solvents such as water, alcohols such as ethanol, butanol, benzyl alcohol, glycerol, hydrocarbons, propylene glycol, polyethylene glycols, N-methylpyrrolidone, and mixtures thereof.

The active ingredients can, where appropriate, also be dissolved in physiologically tolerated vegetable or synthetic oils suitable for injection.

Solubilizers which may be mentioned are: solvents which promote the dissolving of the active ingredient in the main solvent or prevent its precipitation. Examples are polyvinylpyrrolidone, polyethoxylated castor oil, polyethoxylated sorbitan esters.

Preservatives are: benzyl alcohol, trichlorobutanol, p-hydroxybenzoic esters, n-butanol.

Oral solutions are used directly. Concentrates are used orally after previous dilution to the use concentration. Oral solutions and concentrates are prepared as described above for injection solutions, it being possible to dispense with sterile operations.

Solutions for use on the skin are poured on, painted on, rubbed in, sprayed on or applied by dipping, bathing or washing. These solutions are prepared as described above for solutions for injection.

It may be advantageous to add thickeners during preparation. Thickeners are: inorganic thickeners such as bentonites, colloidal silica, aluminium monostearate, organic thickeners such as cellulose derivatives, polyvinyl alcohols and their copolymers, acrylates and methacrylates.

Gels are applied to or spread on the skin or introduced into body cavities. Gels are prepared by mixing solutions which have been prepared as described for solutions for injection with sufficient thickener to result in a clear composition with an ointment-like consistency. The thickeners employed are the thickeners indicated hereinbefore.

Pour-on formulations are poured or sprayed on to limited areas of the skin, in which case the active ingredient either penetrates through the skin and has a systemic action or is distributed on the surface of the body.

Pour-on formulations are prepared by dissolving, suspending or emulsifying the active ingredient in suitable solvents or mixtures of solvents which are compatible with skin. Further excipients such as colorants, absorption-promoting substances, antioxidants, light stabilizers, adhesives are added where appropriate.

Solvents which may be mentioned are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol monobutyl ether, ketones such as acetone, methyl ethyl ketone, aromatic and/or aliphatic hydrocarbons, vegetable or synthetic oils, DMF, dimethylacetamide, N-methylpyrrolidone, 2,2-dimethyl-4-hydroxymethyl-1,3-dioxolane.

Colorants are all colorants which are approved for use on livestock and which can be dissolved or suspended.

Absorption-promoting substances are for example DMSO, spreading oils such as isopropyl myristate, dipropylene glycol pelargonate, silicone oils, fatty acid esters, triglycerides, fatty alcohols.

Antioxidants are sulphites or metabisulphites such as potassium metabisulphite, ascorbic acid, butylated hydroxytoluene, butylated hydroxyanisole, tocopherol.

Examples of light stabilizers are substances from the class of benzophenones or novantisolic acid.

Examples of adhesives are cellulose derivatives, starch derivatives, polyacrylates, natural polymers such as alginates, gelatin.

Emulsions can be used orally, cutaneously or as injections.

Emulsions are either of the water-in-oil type or of the oil-in-water type.

They are prepared by dissolving the active ingredient either in the hydrophobic or in the hydrophilic phase and homogenizing the latter with the solvent of the other phase with the assistance of suitable emulsifiers and, where appropriate, further excipients such as colorants, absorption-promoting substances, preservatives, antioxidants, light stabilizers, viscosity-increasing substances.

Mention may be made of the following as hydrophobic phase (oils): paraffin oils, silicone oils, natural vegetable oils such as sesame oil, almond oil, castor oil, synthetic triglycerides such as caprylic/capric acid biglyceride, triglyceride mixture with vegetable fatty acids of chain length C₈₋₁₂ or other specially selected natural fatty acids, partial glyceride mixtures of saturated or unsaturated, possibly also hydroxyl group-containing fatty acids, mono- and diglycerides of C₈/C₁₀-fatty acids.

Fatty acid esters such as ethyl stearate, di-n-butyl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric esters of saturated fatty alcohols of chain length C₁₂-C₁₈, isopropyl stearate, oleyl oleate, decyl oleate, ethyl oleate, ethyl lactate, waxy fatty acid esters such as dibutyl phthalate, diisopropyl adipate, ester mixtures related to the latter inter alia. Fatty alcohols such as isotridecyl alcohol, 2-octyldodecanol, cetylstearyl alcohol, oleyl alcohol.

Fatty acids such as, for example, oleic acid and mixtures thereof.

Mention may be made of the following as hydrophilic phase:

water, alcohols such as, for example, propylene glycol, glycerol, sorbitol and mixtures thereof.

Emulsifiers which may be mentioned are:

nonionic surfactants, for example polyethoxylated castor oil, polyethoxylated sorbitan monooleate, sorbitan monostearate, glycerol monostearate, polyoxyethyl stearate, alkylphenol polyglycol ether;
ampholytic surfactants such as di-Na N-lauryl-β-iminodipropionate or lecithin;
anionic surfactants such as Na-lauryl sulphate, fatty alcohol ether sulphates, mono/dialkyl polyglycol ether orthophosphoric ester monoethanolamine salt;
cationic surfactants such as cetyltrimethylammonium chloride.

Further suitable excipients which may be mentioned are:

viscosity-increasing and emulsion-stabilizing substances such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silica or mixtures of the substances mentioned.

Suspensions can be used orally, cutaneously or as injection. They are prepared by suspending the active ingredient in a liquid carrier where appropriate with addition of further excipients such as wetting agents, colorants, absorption-promoting substances, preservatives, antioxidants, light stabilizers.

Liquid carriers which may be mentioned are all homogeneous solvents and solvent mixtures.

Wetting agents (dispersants) which may be mentioned are the surfactants indicated hereinbefore.

Further excipients which may be mentioned are those indicated hereinbefore.

Semisolid preparations can be administered orally or cutaneously. They differ from the suspensions and emulsions described above only through their higher viscosity.

Solid preparations are produced by mixing the active ingredients with suitable carriers, where appropriate with the addition of excipients, and bringing to the desired shape.

Carriers which may be mentioned are all physiologically tolerated inert solids. Inorganic and organic substances serve as such. Examples of inorganic substances are sodium chloride, carbonates such as calcium carbonate, hydrogencarbonates, aluminas, silicas, clays, precipitated or colloidal silicon dioxide, phosphates.

Examples of organic substances are sugars, cellulose, foodstuffs and feedstuffs such as milk powder, animal meals, ground and crushed grains, starches.

Excipients are preservatives, antioxidants, colorants, which have already been mentioned hereinbefore.

Further suitable excipients are lubricants and glidants such as, for example, magnesium stearate, stearic acid, talc, bentonite, disintegration-promoting substances such as starch or crosslinked polyvinylpyrrolidone, binders such as, for example, starch, gelatin or linear polyvinylpyrrolidone, and dry binders such as microcrystalline cellulose.

The active ingredients may be present in combination with synergists or with further active ingredients.

Examples of further active ingredients which are suitable are:

coccidiostats such as robenidine or amprolium, in some cases in combination with folic acid antagonists;
polyether antibiotics such as monensin, salinomycin, lasalocid, narasin, semduramicin or in particular maduramicin;
triazinones such as toltrazuril, ponazuril or diclazuril;
sulphonamides;
anthelmintics, e.g. febantel, benzimidazole anthelmintics or depsipeptide anthelmintics such as PF 1022 A or emodepside.

Preparations ready for use comprise the active ingredients in each case in concentrations of from 0.005 ppm to 50 ppm, preferably from 0.1 to 10 ppm.

It has generally proved advantageous to administer amounts of about 0.05 to about 50 mg, preferably 0.1 to 20 mg, of active ingredient per kg of body weight and per day to achieve effective results.

Mixed with other remedies for coccidiosis or polyether antibiotics, the active ingredients according to the invention are present in the ratio 1:0.01-50 to 1:1-50.

The active ingredients can also be administered together with the feed or drinking water to the stock.

Feedstuffs and foodstuffs comprise 0.005 to 250 ppm, preferably 0.05 to 100 ppm of the active ingredient in combination with a suitable edible material.

A feedstuff and foodstuff of this type can be administered both for curative purposes and for prophylactic purposes.

A feedstuff or foodstuff of this type is produced by a concentrate or a premix which comprises 0.5 to 30%, preferably 1 to 20% by weight of an active ingredient mixed with an edible organic or inorganic carrier being mixed with conventional feedstuffs. Examples of edible carriers are maize flour or maize and soya flour or mineral salts, which preferably comprise a small amount of an edible dust-preventing oil, e.g. maize oil or soya oil. The premix obtained in this way can then be added to the complete feedstuff before it is fed to the stock.

The use for histomoniasis may be described by way of example:

for the cure and prophylaxis of histomoniasis in poultry, especially in chickens, ducks, geese or turkey cocks, 0.005 to 100 ppm, preferably 0.05 to 100 ppm, of an active ingredient is mixed with a suitable edible material, e.g. a nutritious feedstuff. If desired, these amounts can be increased, especially if the active ingredient is well tolerated by the recipient. Administration via the drinking water can take place correspondingly.

It may nevertheless occasionally be necessary to deviate from the stated amounts, in particular as a function of the body weight of the experimental animal or the type of administration method, but also because of the genus of animal and its individual response to the active ingredient or the mode of formulation and the time or interval at which it is administered. Thus, it may suffice in certain cases to make do with less than the aforementioned minimum amount, whereas in other cases the stated upper limit must be exceeded. It may be expedient on administration of larger quantities to divide these into a plurality of single administrations over the course of the day.

The efficacy of the compounds according to the invention can be demonstrated for example in cage trials with the following trial design in which the stock are treated with the respective active ingredient.

An active ingredient-containing feed is prepared in such a way that the necessary amount of active ingredient is thoroughly mixed with an animal feed which is balanced in terms of nutrients, e.g. with the chick feed indicated below.

If the intention is to prepare a concentrate or a premix which is eventually intended to be diluted in the feed to the values mentioned in the trial, in general about 1 to 30%, preferably about 10 to 20% by weight of active ingredient are mixed with an edible organic or inorganic carrier, e.g. maize and soya meal or mineral salts which comprise a small amount of an edible antidust oil, e.g. maize oil or soya oil. The premix obtained in this way can then be added to the complete poultry feed before administration.

An example of a suitable composition for use of the substances according to the invention in poultry feed is as follows.

| | |
|---|---|
| 52.00% | crushed feed grains, specifically 40% maize, 12% wheat |
| 17.00% | extr. soya meal |
| 5.00% | maize gluten feed |
| 5.00% | wheat feed flour |
| 3.00% | fish meal |
| 3.00% | mineral mix |
| 3.00% | lucerne-grass meal |
| 2.50% | vitamin premix |
| 2.00% | wheat germ, crushed |
| 2.00% | soya oil |
| 2.00% | fish-bone meal |
| 1.50% | whey powder |
| 1.00% | molasses |
| 1.00% | brewer's yeast bound to Brewer's grains |
| 100.00% | |

Such a feed comprises 18% crude protein, 5% crude fibre, 1% Ca, 0.7% P, and per kg 1200 I.U. vitamin A, 1200 I.U. vitamin D3, 10 mg vitamin E, 20 mg zinc bacitracin.

### BIOLOGICAL EXAMPLE

Young, susceptible turkey cock chicks were kept in cages and underwent intracloacal infection in a conventional way at an age of 2 weeks with about 5 log 10 infectious Histomonas meleagridis microorganisms. With the test groups which had been treated with the test compounds, two control groups were kept for comparison purposes (infected/untreated and uninfected/untreated). The test groups consisted of 6 to 10 turkeys. The test compounds, either the compound of the formula (I-1) or (I-2), were administered to the treated test groups 1 day before the infection and 13 days thereafter, with the feed. At the end of the trial, the animals were sacrificed and examined for lesions typical of histomoniasis (“blackhead disease”). The results of the two trials are summarized in Tables 1 and 2.

TABLE 1

| Group | Test 1 | Weight gain* |
|---|---|---|
| 1 | 10 ppm compound (I-2) in the feed | 35 |
| 2 | 5 ppm compound (I-1) in the feed | 61 |
| 3 | Infected/untreated control | 7 |
| 4 | Uninfected/untreated control | 100 |

*weight gain of group 4 was set at 100%

TABLE 2

| | | Liver | Caecum |
|---|---|---|---|
| Group | Test 2 | lesions* | lesions* |
| 1 | 10 ppm compound (I-2) in the feed | 0 | 2.1 |
| 1 | 30 ppm compound (I-2) in the feed | 0 | 0.8 |
| 2 | 5 ppm compound (I-1) in the feed | 0.8 | 3.5 |
| 2 | 15 ppm compound (I-1) in the feed | 0 | 3 |
| 3 | Infected/untreated control | 1.8 | 3.8 |
| 4 | Uninfected/untreated control | 0 | 0 |

*assessment scale from 0 (no lesions), 1, 2, 3 to 4 (most severe lesions)

It was possible to show that the test compounds are clinically effective against the effects of Histomonas meleagridis infection. In Test 1, Histomonas meleagridis caused severe growth impairments and the test compounds weakened these effects.

In Test 2, Histomonas meleagridis caused distinct liver and caecum lesions with subsequent blackhead disease. Both test compounds reduced these liver lesions, which cause the disease, markedly. The compound of the formula (I-2) also reduced the caecum lesions.

1.-6. (canceled)

7. A method for controlling histomoniasis in poultry comprising, administering an effective amount of a benzimidazole of the formula (I) to the poultry in need thereof 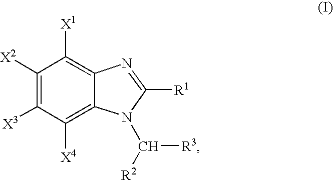
in which
R¹ is fluoroalkyl,
R² is hydrogen or alkyl,
R³ is a radical of the formula 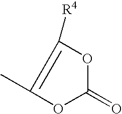
or is a radical of the formula 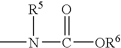
R⁴ is alkyl,
R⁵ is alkyl or substituted phenyl,
R⁶ is alkyl,
X¹, X², X³ and X⁴ are independently of one another hydrogen, halogen, haloalkyl, haloalkoxy, haloalkylthio or haloalkylsulphonyl,
or else
X² and X³ or X³ and X⁴ together are a dioxyhaloalkylene radical.

8. The method of claim 7, wherein
R¹ is C₁-C₄-fluoroalkyl,
R² is hydrogen or C₁-C₄-alkyl,
R⁴ is C₁-C₄-alkyl,
R⁵ is C₁₋₆-alkyl or phenyl which is optionally substituted one or more times by C₁₋₄-alkyl, C₁₋₄-haloalkyl, halogen, nitro, C₁₋₄-alkoxy, C₁₋₄-haloalkoxy or optionally mono- or poly-halogen-substituted methylene- or ethylenedioxy,
R⁶ is C₁₋₄-alkyl,
X¹, X², X³ and X⁴ are independently of one another hydrogen, F, Cl, Br, C₁-C₄-haloalkyl, C₁-C₄-haloalkoxy, C₁-C₄-haloalkylthio, C₁-C₄-haloalkylsulphonyl, or
X² and X³ or X³ and X⁴ together are a dioxyhalo-C₁-C₄-alkylene radical.

9. A method for controlling histomoniasis in poultry comprising, administering an effective amount of a benzimidazole of the formula (I-1) 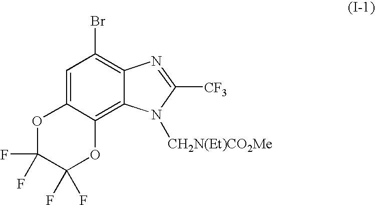
to the poultry in need thereof.

10. A method for controlling histomoniasis in poultry comprising, administering an effective amount of a benzimidazole of the formula (I-2) 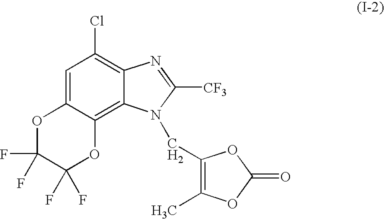
to the poultry in need thereof.

11. The method of claim 1, wherein the poultry is a turkey.

12. The method of claim 1, wherein the effective amount is from 0.05 to 50 mg of the benzimidazole per kg of body weight of the poultry.
